(19) United States
(12) Patent Application Publication
(10) Pub. No.: US 2024/0399206 A1
(43) Pub. Date: Dec. 5, 2024

(54) ELECTRONIC DEVICE, DISPLAY METHOD, AND STORAGE MEDIUM

(71) Applicant: CASIO COMPUTER CO., LTD., Tokyo (JP)

(72) Inventors: Tomoharu YAMAGUCHI, Tokyo (JP); Hiroyasu IDE, Tokyo (JP)

(73) Assignee: CASIO COMPUTER CO., LTD., Tokyo (JP)

(21) Appl. No.: 18/663,126

(22) Filed: May 14, 2024

(30) Foreign Application Priority Data
May 30, 2023 (JP) 2023-088531

(51) Int. Cl. A63B 24/00; A63B 71/06; G01C 22/00

(52) U.S. Cl. CPC A63B 24/0062; A63B 71/0622; G01C 22/006; A63B 2071/0663; A63B 2220/12; A63B 2220/34; A63B 2220/40; A63B 2220/62; A63B 2220/836; A63B 2230/62

(57) ABSTRACT

An electronic device includes a memory that stores a program; and at least one processor that executes the program. The processor is configured to, derive a pitch for each step of a user based on data acquired from a predetermined sensor, derive a difference between a first left foot statistical value of the pitch derived for each step in a predetermined number of steps of the left foot, and a first right foot statistical value of the pitch derived for each step in the predetermined number of steps of the right foot, display a value of the derived difference on a display, and update the displayed value to the value that is newly derived and display the newly derived value in a case in which it is determined that each of a second left foot statistical value and a second right foot statistical value satisfy a predetermined condition.

### REFERENCE TO RELATED APPLICATIONS

The present application claims priority under Paris Convention to Japanese Patent Application No. 2023-088531, filed on 30th of May 2023, the disclosure of which, including description, claims, abstract and drawings, is incorporated herein by reference in its entirety.

### TECHNICAL FIELD

The present disclosure relates to an electronic device, a display method, and a storage medium.

### BACKGROUND OF THE INVENTION

Conventionally, electronic devices that measure and display exercise information during walking or running are known. For example, Japanese Unexamined Patent Publication No. 2017-6371 describes an exercise support device that measures and displays elapsed time (pitch) between landing timings of left and right feet during running.

There is also a device that presents (displays) a difference (left/right pitch difference) between elapsed time from a left foot landing timing to a right foot landing timing and the elapsed time from the right foot landing timing to the left foot landing timing.

### SUMMARY OF THE INVENTION

In order to solve the above problems, the electronic device of the present invention includes:
a memory that stores a program; and
at least one processor that executes the program,
wherein the processor is configured to,
derive a pitch for each step of a user based on data acquired from a predetermined sensor, wherein the pitch is derived for each of a left foot and a right foot,
derive a difference between a first left foot statistical value of the pitch derived for each step in a predetermined number of steps of the left foot, and a first right foot statistical value of the pitch derived for each step in the predetermined number of steps of the right foot,
display a value of the derived difference on a display, and
update the displayed value of the difference to the value of the difference that is newly derived and display the newly derived value in a case in which it is determined that each of a second left foot statistical value regarding a pitch derived for each step in the predetermined number of steps of the left foot and a second right foot statistical value of the pitch derived for each step in the predetermined number of steps of the right foot satisfy a predetermined condition.

### BRIEF DESCRIPTION OF DRAWINGS

FIG. 1A is a diagram illustrating a user wearing an electronic device according to an embodiment of the present disclosure.

FIG. 1B is a diagram showing an outer appearance of the electronic device.

FIG. 2 is a block diagram showing a functional configuration of the electronic device shown in FIG. 1B.

FIG. 3 is a flowchart showing a flow of a gait analysis process executed by a controller shown in FIG. 2.

FIG. 4A is a diagram showing an example of data storage in a pitch derivation result storage memory.

FIG. 4B is a diagram showing a maximum value, a minimum value, and a range of the recent five steps in a case where the pitch shown in FIG. 4A is acquired as the pitch of the recent five steps.

FIG. 5A is a diagram showing an example of data storage in a pitch derivation result storage memory.

FIG. 5B is a diagram showing a maximum value, a minimum value, and a range of the recent five steps in a case where the pitch shown in FIG. 5A is acquired as the pitch of the recent five steps.

FIG. 6A is a diagram showing a change in a display on a display 130 when a reliability of the measured pitch is high.

FIG. 6B is a diagram showing a change in a display on the display 130 when the reliability of the measured pitch is low.

### DETAILED DESCRIPTION OF THE INVENTION

Hereinafter, embodiments to implement the present disclosure are described with reference to the drawings. However, various limitations that are technically preferable to execute the present disclosure are described in the embodiments below. Therefore, the technical scope of the present disclosure is not limited to the embodiments described below and the illustrated examples.

### [Configuration of Electronic Device 100]

An electronic device 100 according to the present embodiment measures and displays a user's exercise index during walking.

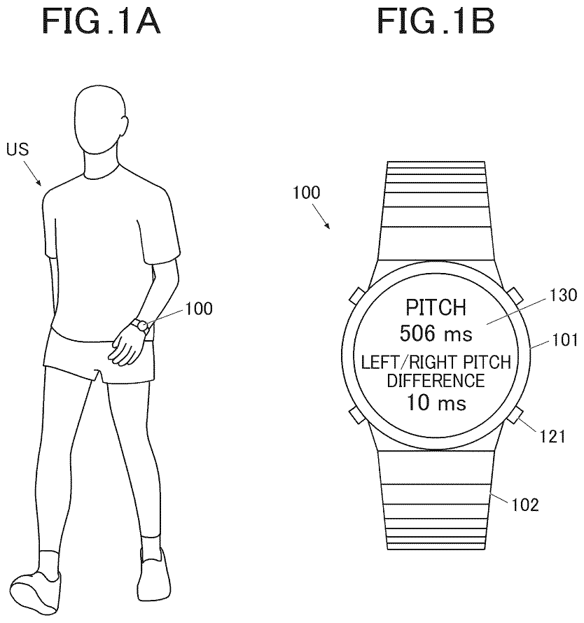

The electronic device 100 is worn on a wrist of a user US, for example, as shown in FIG. 1A. As shown in FIG. 1A, the electronic device 100 has a wristwatch-type outer appearance, and includes the following components that can be roughly divided into a device main body 101 equipped with a display 130 (display) for presenting predetermined information to the user US, and a belt 102 for attaching the device main body 101 to the wrist of the user US by wrapping it around the wrist of the user US.

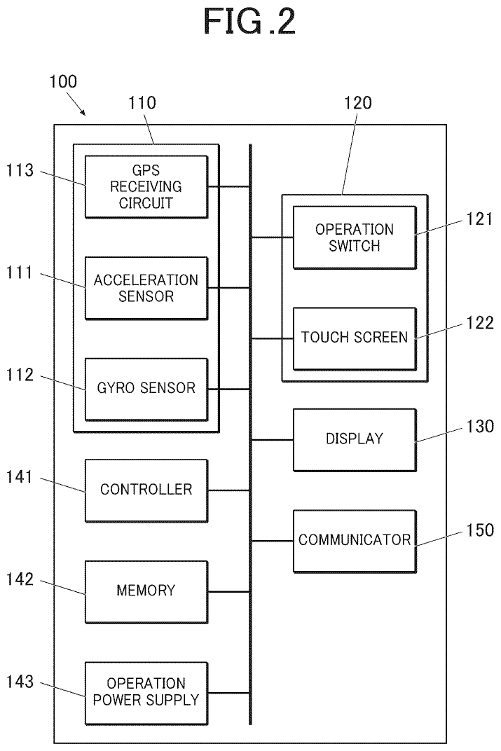

Specifically, as shown in FIG. 2, the electronic device 100 roughly includes a sensor section (detecting section) 110, an operator 120, a display 130, a controller 141 (processor), a memory (storage) 142, an operation power supply 143, and a communicator 150.

As shown in FIG. 2, the sensor section 110 includes an acceleration sensor 111, a gyro sensor (angular velocity sensor) 112, and a GPS receiving circuit (position sensor) 113.

The acceleration sensor 111 detects the rate of change (acceleration) in the movement speed of the user US during exercise, and outputs the detected acceleration data to the controller 141. The gyro sensor 112 detects the change in the movement direction (angular velocity) of the user US during exercise, and outputs the detected angular velocity data to the controller 141. Then, by executing a predetermined control program, the controller 141 derives (calculates) the exercise index such as a walking pitch and a left/right pitch difference based on a waveform indicating the temporal change of the acceleration data detected by the acceleration sensor 111 and/or the angular velocity data detected by the gyro sensor 112.

Here, in the present disclosure, the pitch is the time between a left foot (right foot) landing timing and a right foot (left foot) landing timing during walking (or running) (that is, the time required for one step). The left/right pitch difference is the difference between the time from the left foot landing timing to the right foot landing timing (left pitch) and the time from the right foot landing timing to the left foot landing timing (right pitch). According to the present embodiment, the “left pitch-right pitch” is to be the left/right pitch difference but the “right pitch—left pitch” may be the left/right pitch difference.

Furthermore, the GPS receiving circuit 113 detects (geographical) position data consisting of latitude and longitude by receiving radio waves from a plurality of GPS satellites. Based on the position data detected by the GPS receiving circuit 113, the controller 141 obtains the travel distance. Furthermore, the GPS receiving circuit 113 uses the Doppler shift effect of radio waves from GPS satellites to obtain the moving speed of the user US.

Note that the sensor section 110 only needs to include at least the acceleration sensor 111, and may be configured without the gyro sensor 112 and the GPS receiving circuit 113.

As shown in FIG. 2, the operator 120 includes an operation switch 121 and a touch screen 122. The operation switch 121 includes a button switch, for example, as shown in FIG. 1B, and is used for operations such as starting, ending, changing, and pausing an operation mode, and various settings.

Further, the touch screen 122 is arranged on the front surface of the display 130 or is formed integrally with the front surface of the display 130, and detects a touched position on the display 130 and outputs the detected position to the controller 141. Note that the operator 120 may include both the operation switch 121 and the touch screen 122, or may include only one of them.

The display 130 has a display device such as a liquid crystal display panel or an organic EL display panel, and displays various types of information derived based on data (sensor data) acquired by the sensor section 110 described above at least during the exercise of the user US. On the display 130, for example, as shown in FIG. 1B, character information including numerical values such as the pitch and the left/right pitch difference is displayed.

The controller 141 is a computer and includes at least one central processing unit (CPU), a read only memory (ROM), a random access memory (RAM), and the like. The controller 141 controls the sections of the electronic device 100. Specifically, the CPU of the controller 141 reads a designated program from among the system program and various application programs stored in the ROM, deploys it in the RAM, and performs various processing in cooperation with the deployed program.

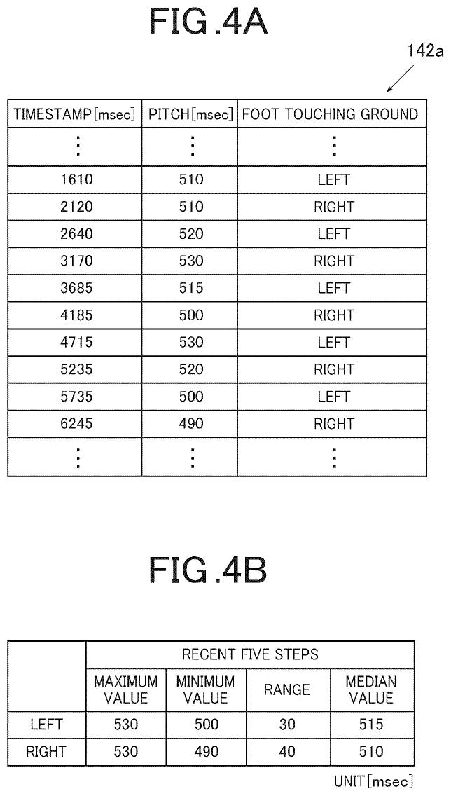

The memory 142 is composed of a nonvolatile memory or the like. The memory 142 mutually associates numerical information of exercise indices such as pitch during walking and left/right pitch difference during walking, which is derived based on sensor data such as acceleration data acquired by the sensor section 110 described above, with elapsed time since the start of walking. For example, the memory 142 includes a pitch derivation result storage memory 142a shown in FIG. 4A.

The operation power supply 143 supplies driving power to each component in the electronic device 100. The operation power supply 143 includes primary batteries such as commercially available coin-type batteries and button-type batteries, secondary batteries such as lithium-ion batteries and nickel-metal hydride batteries, as well as power supply using energy harvesting techniques that generate electricity using energy such as vibration, light, heat, and electromagnetic waves (energy harvesting).

The communicator 150 performs communication control for communicating with an external device using a wireless LAN (Local Area Network) such as Wi-Fi, a mobile phone communication network, or Bluetooth (registered trademark).

### [Operation of Electronic Device 100]

Next, the operation of the electronic device 100 according to the present embodiment is described.

When the user US operates the operator 120 to instruct transition to a gait analysis mode, the controller 141 starts the gait analysis process.

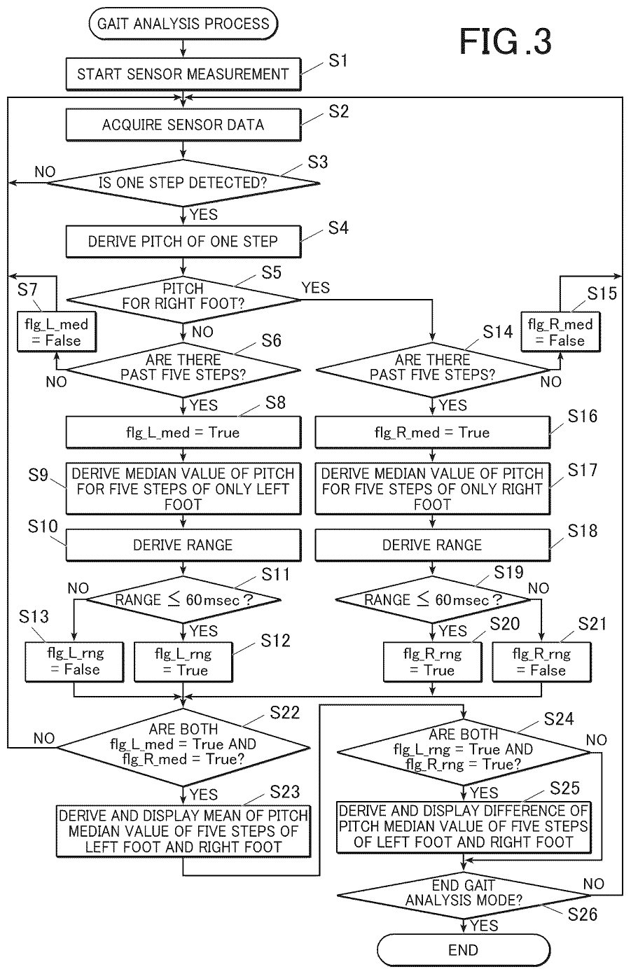

FIG. 3 is a flowchart showing a flow of the gait analysis process. The gait analysis process is performed by the CPU of the controller 141 in coordination with the program stored in the ROM.

First, the controller 141 starts time measurement and starts measurement (data acquisition) by the sensor section 110 (step S1). Then, the controller 141 acquires data (sensor data) from the sensor section 110, associates the time (timestamp: elapsed time from the start of walking) and stores the data in the memory 142 (step S2).

Next, the controller 141 analyzes the sensor data acquired from the sensor section 110 and determines whether a step has been detected (step S3).

For example, the controller 141 analyzes the acceleration data from the acceleration sensor 111, and if a landing impact can be detected, the controller 141 determines that the foot touching the ground is detected, that is, one step is detected. Note that the method of detecting the landing impact from the acceleration data may be any known method and is not particularly limited.

If it is determined that the one step is not detected (step S3; NO), the controller 141 returns the process to step S2.

If it is determined that the one step is detected (step S3; YES), the controller 141 derives the pitch of the one step (step S4).

The controller 141 derives as the pitch the difference between the time when the previous impact was detected (i.e., the time when the foot touched the ground last time) and the time when the current impact was detected (i.e., the time when the foot touched the ground this time). The derived result of the pitch is stored in the pitch derivation result storage memory 142a (see FIG. 4A) in association with the time (timestamp).

Next, the controller 141 determines whether the derived pitch is the pitch of the right foot (step S5).

Here, in the electronic device 100, before the user US starts using the electronic device 100, it is set in advance whether the electronic device 100 is to be worn on the left (left wrist) or the right (right wrist). The memory 142 stores the setting information (left and right setting information). For example, the controller 141 determines which arm was swinging forward at the time of the current impact detection, based on the left and right setting information, acceleration data from the acceleration sensor 111, and angular velocity data from the gyro sensor 112. Based on the result of the judgment, it is determined whether the left or right foot is in contact with the ground when the impact is detected. If it is determined that the left foot is in contact with the ground, it is determined that the pitch is that of the left foot (left pitch), and if it is determined that the right foot is in contact with the ground, it is determined that it is the pitch of the right foot (right pitch). The foot that touched the ground is stored in the pitch derivation result storage memory 142a shown in FIG. 4A in association with the pitch derivation result.

If it is determined that the derived pitch is not the pitch of the right foot (step S5; NO), that is, if it is determined that the derived pitch is the pitch of the left foot, the controller 141 determines whether information on the pitch of the left foot for five steps exists in the pitch derivation result storage memory 142a (step S6). Here, according to the present embodiment, a median value (first left foot statistical value, first right foot statistical value) and range (second left foot statistical value, second right foot statistical value) of the pitch of the latter stage are derived using pitch information for five steps. The median value or the range of the pitch is not derived until the pitch information for five steps is acquired. However, the number of data used for the purpose of deriving the median value and the range of the pitch in the latter stage is not particularly limited as long as it is plural.

If it is determined that the information on the pitch of the left foot for five steps does not exist in the pitch derivation result storage memory 142a (step S6; NO), the controller 141 sets a variable flg_L_med to False (step S7), and returns to step S2.

If it is determined that the information on the pitch of the left foot for five steps exists in the pitch derivation result storage memory 142a (step S6; YES), the controller 141 sets a variable flg_L_med to True (step S8).

Next, the controller 141 derives the median value of the pitch of the recent five steps of the left foot (step S9).

Next, the controller 141 derives a range which is a difference between the maximum value and the minimum value of the pitch in the recent five steps of the left foot (step S10), and determines whether it is the range≤60 msec (step S11). Note that although the threshold value for comparison with the derived range is set to 60 msec here, the value is not limited to this.

If it is determined that it is the range≤60 msec (step S11; YES), the controller 141 sets the variable flg_L_rng to True (step S12), and proceeds to step S22.

If it is determined that it is not the range≤60 msec (step S11; NO), the controller 141 sets the variable flg_L_rng to False (step S13), and proceeds to step S22.

In step S5, if it is determined that the derived pitch is the pitch of the right foot (step S5; YES), the controller 141 determines whether information on the pitch of the right foot for five steps exists in the pitch derivation result storage memory 142a (step S14).

If it is determined that the information on the pitch of the right foot for five steps does not exist in the pitch derivation result storage memory 142a (step S14; NO), the controller 141 sets a variable flg_R_med to False (step S15), and returns to step S2.

In step S14, if it is determined that the information on the pitch of the right foot for five steps exists in the pitch derivation result storage memory 142a (step S14; YES), the controller 141 sets a variable flg_R_med to True (step S16).

Next, the controller 141 derives the median value of the pitch of the recent five steps of the right foot (step S17).

Next, the controller 141 derives a range which is a difference between the maximum value and the minimum value of the pitch in the recent five steps of the right foot (step S18), and determines whether it is the range≤60 msec (step S19). Note that although the threshold value for comparison with the derived range is set to 60 msec here, the value is not limited to this.

If it is determined that it is the range≤60 msec (step S19; YES), the controller 141 sets the variable flg_R_rng to True (step S20), and proceeds to step S22.

In step S19, if it is determined that it is not the range ≤60 msec (step S19; NO), the controller 141 sets the variable flg_R_rng to False (step S21), and proceeds to step S22.

In step S22, the controller 141 determines whether it is flg_L_med=True and flg_R_med=True (step S22).

If it is determined that it is not flg_L_med=True and flg_R_med=True (step S22; NO), the controller 141 returns to step S2. That is, if the median pitch of the recent five steps of the left foot or the median pitch of the recent five steps of the right foot has not been derived, the process returns to step S2.

If it is determined that it is flg_L_med=True and flg_R_med=True (step S22; YES), that is, if the median pitch of the recent five steps of the left foot and the median pitch of the recent five steps of the right foot have been derived, the controller 141 derives the mean of the median pitches of the recent five steps of the left foot and right foot (third statistical value, mean of the left and right pitches) as the pitch to be displayed and displays the derived pitch on the display 130 (step S23).

If the pitch is already displayed on the display 130, the controller 141 updates the display of the currently displayed pitch to the display of the newly derived pitch.

Next, the controller 141 determines whether it is flg_L_rng=True and flg_R_rng=True (step S24).

If it is determined that flg_L_rng=True and flg_R_rng=True (step S24; YES), the controller 141 derives the difference between the median values of the pitches of the recent five steps of the left foot and right foot as the left/right pitch difference, and controls the display 130 to display the derived left/right pitch difference (step S25). Then, the process moves to step S26.

If the left/right pitch difference is already displayed on the display 130, the controller 141 updates the display of the currently displayed left/right pitch difference to the display of the newly derived left/right pitch difference.

Note that the controller 141 stores the derived pitch and left/right pitch difference in the memory 142 in association with the elapsed time from the start of walking.

If it is determined that it is not flg_L_rng=True and flg_R_rng-True (step S24; NO), that is, if at least one of flg_L_rng and flg_R_rng is False, the controller 141 does not update the display of the left/right pitch difference and the process moves to step S26.

In step S26, the controller 141 determines whether an instruction to end the gait analysis mode has been given by operating the operator 120 (step S26).

If it is determined that the instruction to end the gait analysis mode has not been given by operating the operator 120 (step S26; NO), the controller 141 returns the process to step S2.

If it is determined that the instruction to end the gait analysis mode has been given by operating the operator 120 (step S26; YES), the controller 141 ends the gait analysis process.

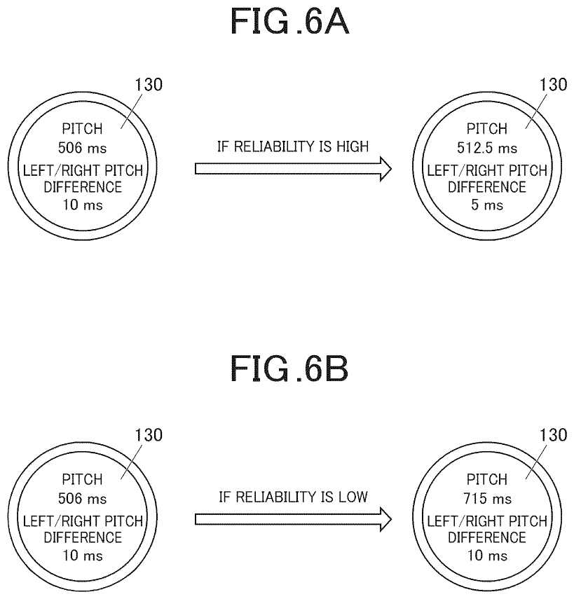

To give a specific example, if the pitch of each of the five recent steps of the left foot and the pitch of each of the five recent steps of the right foot are the values shown in FIG. 4A, as shown in FIG. 4B, the maximum value of the pitch of the five recent steps of the left foot is 530, and the minimum value is 500. Therefore, the pitch range of the recent five steps of the left foot is 30 (msec). Further, since the maximum value of the pitch of the five recent steps of the right foot is 530 and the minimum value is 490, the range of the pitch of the recent five steps of the right foot is 40 (msec). In other words, if the pitch of each of the five recent steps of the left foot and the pitch of each of the five recent steps of the right foot are the values shown in FIG. 4A, the pitch ranges of both feet are both within 60 (msec) and the data is considered to have high reliability. With this, the displayed pitch (third statistical value) and left/right pitch difference are derived. Then, as shown in FIG. 6A, the pitch and the left/right pitch difference displayed on the display 130 are updated to the derived values.

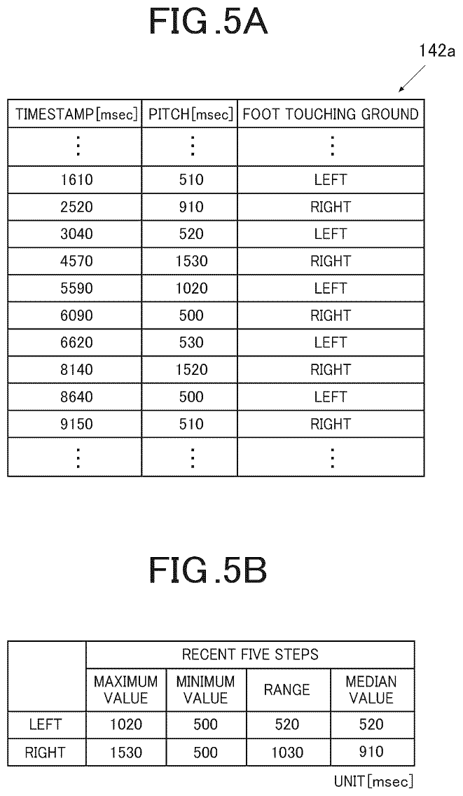

For example, if the pitch of each of the five recent steps of the left foot and the pitch of each of the five recent steps of the right foot are the values shown in FIG. 5A, as shown in FIG. 5B, the maximum value of the pitch of the five recent steps of the left foot is 1020, and the minimum value is 500. Therefore, the pitch range of the recent five steps of the left foot is 520 (msec). Further, since the maximum value of the pitch of the five recent steps of the right foot is 1530 and the minimum value is 500, the range of the pitch of the recent five steps of the right foot is 1030 (msec). In other words, if the pitch of each of the five recent steps of the left foot and the pitch of each of the five recent steps of the right foot are the values shown in FIG. 5A, the pitch ranges of the left foot and the right foot are both larger than 60 (msec) and the data is considered to have low reliability. With this, the left/right pitch difference is not derived and only the displayed pitch (third statistical value) is derived. Then, as shown in FIG. 6B, only the pitch displayed on the display 130 is updated to the newly derived value and the left/right pitch difference is not updated.

In this way, in the above gait analysis process, if the reliability of the measured pitch is low, the display of the left/right pitch difference is not updated. The display of the left/right pitch difference is updated only when the reliability of the measured pitch is high. Therefore, it is possible to improve the accuracy of the displayed left/right pitch difference.

Note that if the range of at least one of the left foot and the right foot exceeds 60 (msec) and the left/right pitch difference displayed on the display 130 is not updated, the controller 141 may display the left/right pitch difference in a manner that enables the user US to identify that the left/right pitch difference is not updated. For example, if the left/right pitch difference has not been updated, the left/right pitch difference is displayed in a different color. Alternatively, a predetermined mark may be added near the display of the left/right pitch difference, or a text may be displayed to indicate that the left/right pitch difference has not been updated.

In addition, in the above-mentioned gait analysis process, the display of the pitch value is updated every step, but if at least one range of the left foot and right foot does not meet the predetermined condition (60 (msec) or less), in other words, if the reliability of the data is low, the display of the pitch value may not be updated also. For example, by performing the process of step S23 in FIG. 3 after the process of step S24, if the reliability of the data is low, the display of the pitch value may not be updated also.

The left/right pitch difference is an important exercise index that indicates the left/right balance in how the body is used during walking or running. However, for example, when a movement is performed such as jumping over a puddle or walking (running) on a bumpy road (a road with relatively many bumps and dents), there is the issue that the accuracy of the displayed left/right pitch difference decreases significantly.

In contrast, the controller 141 of the electronic device 100 in the present embodiment derives the pitch of each step of the user US for each of the left foot and the right foot based on data acquired from the acceleration sensor 111 etc. of the sensor section 110. Then, the controller 141 determines whether the range of the pitch of the recent five steps of the left foot and the range of the pitch of the recent five steps of the right foot each satisfy a predetermined condition. Then, when each range satisfies the predetermined condition, the controller 141 derives the left/right pitch difference, and updates the currently displayed value of the left/right pitch difference to the newly derived value of the left/right pitch difference.

Therefore, if the range of the recent five steps of the left foot and/or right foot does not meet the predetermined conditions, the display of the left/right pitch difference is not updated. Therefore, for example, if the correct pitch cannot be derived due to movement such as jumping over a puddle, it is possible to avoid displaying the left/right pitch difference based on the pitch. Therefore, it is possible to improve the accuracy of the notified left/right pitch difference.

For example, the sensor section 110 includes at least the acceleration sensor, and the controller 141 derives the respective pitches of the left foot and right foot based on data acquired from a predetermined sensor including the acceleration sensor. Therefore, the pitch can be derived using at least the acceleration sensor.

Further, the controller 141 further derives the mean of the median values of the pitches derived for each of the left foot and the right foot, and controls the display 130 to display the derived value as the pitch. Therefore, the user US can also confirm the pitch.

Further, if the range of the pitch of the five recent steps of the left foot and the range of the pitch of the five recent steps of the right foot satisfy a predetermined condition, the controller 141 derives the mean of the median values of the pitch derived from each of the left foot and the right foot as the displayed pitch. Then, the controller 141 updates the currently displayed pitch to the newly derived pitch. Therefore, if the range of the recent five steps of the left foot and/or the right foot does not satisfy a predetermined condition, the pitch display is not updated, so the accuracy of the displayed pitch can be improved.

If at least one of the range for the pitch of the recent five steps of the left foot and the range for the pitch of the recent five steps of the right foot does not satisfy a predetermined condition and the value of the left/right pitch difference displayed on the display 130 is not updated, the controller 141 controls the display 130 to display that the left/right pitch difference has not been updated in a manner that is recognizable to the user US.

Therefore, the user US can recognize that the display of the left/right pitch difference has not been updated.

The described contents according to the above embodiments are merely a suitable example of the electronic device, presentation method, and the storage medium regarding the present disclosure, and the present disclosure is not limited to the above.

For example, in the above embodiment, the case where the pitch and the left/right pitch difference during walking are derived and displayed is described. Alternatively, the present invention is also applicable to the case where the pitch and the left/right pitch difference during jogging or the like are derived and displayed.

In addition, in the above embodiment, the difference between the median value (first left foot statistical value) of the pitch derived for each of the recent five steps of the left foot, and the median value (first right foot statistical value) of the pitch derived for each of the recent five steps of the right foot was used as the left/right pitch difference, but the mean value may be used instead of the median value. Further, the number of steps for deriving the median value and mean value is not limited to five steps.

In addition, the mean of the median value of the pitch derived for each of the recent five steps of the left foot, and the median value of the pitch derived for each of the recent five steps of the right foot was displayed as the pitch, but the mean value may be used instead of the median value. Further, the number of steps for deriving the median value and mean value is not limited to five steps.

Furthermore, in the embodiment described above, the electronic device 100 that displays the pitch and the left/right pitch difference is configured to include a predetermined sensor, such as an acceleration sensor or a gyro sensor, for acquiring data for deriving the pitch and the left/right pitch difference. However, the electronic device and the sensor that display the pitch and the left/right pitch difference may be separate bodies.

Further, in the above embodiment, the predetermined sensor for acquiring data for deriving the pitch and the left/right pitch difference is an acceleration sensor or a gyro sensor of the electronic device 100 attached to the arm (wrist) of the user US. However, the predetermined sensor is not limited thereto. For example, the predetermined sensor may be attached to other parts of the user US, such as the upper arm, waist, or ankle of the user US. Moreover, the predetermined sensor does not need to be attached to the user US. For example, the predetermined sensor may be an image sensor (camera). For example, the image sensor may be used to photograph the user US walking or running on a treadmill, and the pitch of each step of the user US may be derived from the obtained photographed image.

Further, in the above embodiment, the pitch and the left/right pitch difference are presented by displaying them on the display 130, but the present invention is not limited to this. For example, the above may be presented by audio.

According to the above embodiments, a ROM is used as the computer readable storage medium storing the program regarding the above disclosure but the examples are not limited to the above. As the computer readable medium, a hard disk, a SSD, a portable recording/storage medium, such as a CD-ROM, can also be used. A carrier wave is also applied as a medium providing the program data according to the present invention via a communication line.

In addition to the above, detailed configurations and detailed operation of the components of the electronic device can also be appropriately modified without departing from the scope of the present invention.

Although the embodiments of the invention have been described above, the technical scope of the invention is not limited to the embodiments described above, but is defined based on the scope of the claims. Furthermore, the technical scope of the invention includes equivalents in which modifications that are not related to the essence of the invention are added to the scope of the claims.

1. An electronic device comprising:
a memory that stores a program; and
at least one processor that executes the program,
wherein the processor is configured to,
derive a pitch for each step of a user based on data acquired from a predetermined sensor, wherein the pitch is derived for each of a left foot and a right foot,
derive a difference between a first left foot statistical value of the pitch derived for each step in a predetermined number of steps of the left foot, and a first right foot statistical value of the pitch derived for each step in the predetermined number of steps of the right foot,
display a value of the derived difference on a display, and
update the displayed value of the difference to the value of the difference that is newly derived and display the newly derived value in a case in which it is determined that each of a second left foot statistical value regarding a pitch derived for each step in the predetermined number of steps of the left foot and a second right foot statistical value of the pitch derived for each step in the predetermined number of steps of the right foot satisfy a predetermined condition.

2. The electronic device according to claim 1, wherein the processor is configured to not update the displayed value of the difference in a case in which it is determined that each of a second left foot statistical value regarding a pitch derived for each step in the predetermined number of steps of the left foot and a second right foot statistical value of the pitch derived for each step in the predetermined number of steps of the right foot does not satisfy a predetermined condition.

3. The electronic device according to claim 1, wherein,
the predetermined sensor includes at least an acceleration sensor, and
the processor is configured to derive the pitch of each of the left foot and the right foot based on data acquired from a predetermined sensor including the acceleration sensor.

4. The electronic device according to claim 1, wherein the processor is configured to,
further derive a third statistical value regarding the pitch, and
display the derived third statistical value on the display.

5. The electronic device according to claim 4, wherein the processor is configured to derive the third statistical value, update the presently displayed third statistical value to the newly derived third statistical value, and display the newly derived third statistical value in a case in which each of the second left foot statistical value and the right foot statistical value satisfy the predetermined condition.

6. The electronic device according to claim 1, wherein the first left foot statistical value and the first right foot statistical value are a median value or a mean value.

7. The electronic device according to claim 1, wherein the second left foot statistical value and the second right foot statistical value are a difference between a maximum value and a minimum value.

8. The electronic device according to claim 1, wherein the processor is to make an output in a manner that it is possible to recognize that the value of the difference is not updated in a case in which the displayed value of the difference is not updated due to at least one of the second left foot statistical value and the second right foot statistical value not satisfying the predetermined condition.

9. A display method comprising:
deriving a pitch for each step of a user based on data acquired from a predetermined sensor, wherein the pitch is derived for each of a left foot and a right foot;
deriving a difference between a first left foot statistical value of the pitch derived for each step in a predetermined number of steps of the left foot, and a first right foot statistical value of the pitch derived for each step in the predetermined number of steps of the right foot;
displaying the derived value of the difference; and
updating the displayed value of the difference to the value of the difference that is newly derived and displaying the newly derived value in a case in which it is determined that each of a second left foot statistical value regarding a pitch derived for each step in the predetermined number of steps of the left foot and a second right foot statistical value of the pitch derived for each step in the predetermined number of steps of the right foot satisfy a predetermined condition.

10. The display method according to claim 9, wherein the displayed value of the difference is not updated in a case in which it is determined that each of a second left foot statistical value regarding a pitch derived for each step in the predetermined number of steps of the left foot and a second right foot statistical value of the pitch derived for each step in the predetermined number of steps of the right foot does not satisfy a predetermined condition.

11. The display method according to claim 9, wherein,
the sensor includes at least an acceleration sensor, and
the pitch of each of the left foot and the right foot is derived based on data acquired from a predetermined sensor including the acceleration sensor.

12. The display method according to claim 9, further comprising,
deriving a third statistical value regarding the pitch, and
displaying the derived third statistical value.

13. The display method according to claim 12, wherein the third statistical value is derived, the presently displayed third statistical value is updated to the newly derived third statistical value and the newly derived third statistical value is displayed in a case in which each of the second left foot statistical value and the right foot statistical value satisfy the predetermined condition.

14. The display method according to claim 9, wherein the first left foot statistical value and the first right foot statistical value are a median value or a mean value.

15. The display method according to claim 9, wherein the second left foot statistical value and the second right foot statistical value are a difference between a maximum value and a minimum value.

16. The display method according to claim 9, wherein an output is performed in a manner that it is possible to recognize that the value of the difference is not updated in a case in which the displayed value of the difference is not updated due to at least one of the second left foot statistical value and the second right foot statistical value not satisfying the predetermined condition.

17. A non-transitory computer-readable storage medium storing a program executed by a computer to perform,
deriving a pitch for each step of a user based on data acquired from a predetermined sensor, wherein the pitch is derived for each of a left foot and a right foot;
deriving a difference between a first left foot statistical value of the pitch derived for each step in a predetermined number of steps of the left foot, and a first right foot statistical value of the pitch derived for each step in the predetermined number of steps of the right foot;
displaying the derived value of the difference on a display; and
updating the displayed value of the difference to the value of the difference that is newly derived and displaying the newly derived value in a case in which it is determined that each of a second left foot statistical value regarding a pitch derived for each step in the predetermined number of steps of the left foot and a second right foot statistical value of the pitch derived for each step in the predetermined number of steps of the right foot satisfy a predetermined condition.
